(19) United States
(12) Patent Application Publication
(10) Pub. No.: US 2013/0280821 A1
(43) Pub. Date: Oct. 24, 2013

(54) METHOD FOR DETECTING AN INFECTION BY HEPATITIS B VIRUS

(71) Applicants: Daniela HUZLY, Schallstadt (DE); Michael Nassal, March (DE)

(72) Inventors: Daniela HUZLY, Schallstadt (DE); Michael Nassal, March (DE)

(73) Assignee: Universitaetsklinikum Freiburg, Freiburg (DE)

(21) Appl. No.: 13/450,010

(22) Filed: Apr. 18, 2012

(51) Int. Cl. G01N 33/576

(52) U.S. Cl. 436/501

(57) ABSTRACT

An immunological confirmation method is disclosed for the detection of hepatitis B virus infection wherein the testing of certain samples showing unclear reactivity is repeated once without and once in the presence of recombinantly produced HBcAg particles. If the sample is truly hepatitis B virus core antibody positive, the rHBcAg will trap the anti-HBcAg antibodies and influence the readout accordingly.

### BACKGROUND TO THE PRESENT INVENTION

Hepatitis B virus (HBV), a small double stranded DNA virus, can cause a wide spectrum of clinical presentations: asymptomatic carrier state, acute self-limited hepatitis, fulminant hepatitis, and chronic liver diseases including chronic hepatitis, liver cirrhosis, and hepatocellular carcinoma. It has a circular genome of 3182 to 3221 base pairs (bp). The outer envelope of the virus comprises the hepatitis B virus surface antigen (HBsAg). The inner capsid having a diameter of about 30 to 35 nm is formed by subunits of hepatitis B virus core antigen (HBcAg).

Four major subtypes have been identified and can be differentiated by antibodies that recognize the different epitopes on the HBV surface. The HBsAg particles carry the common determinant, “a”, as well as d or y and w or r subtype determinants, and are classified into the four major subtypes, i.e., adw, adr, ayw and ayr. Rare sera contain HBsAg particles with all four-subtype determinants (adywr). The antigen determinants for the main HBV subtypes: adw, adr, ayw and ayr lie in the surface or “S” polypeptide. The virus has a high rate of mutation relative to other DNA viruses due to its mode of replication by reverse transcriptase of its pregenomic RNA.

The hepatitis B virus is transferred by blood, blood products and sexual intercourse. The percentage of persons infected with hepatitis B virus changes from country to country. In highly industrialized countries the hepatitis B virus is comparatively rare. In developing countries 20 up to 80% of the population may be infected with hepatitis B virus. Since the virus can easily be transferred by blood and blood products it is important for blood banks to test reliably whether the blood donations are clearly HBV negative or not. The testing of blood samples is performed with a high degree of automatization whereby the testing machines have very high throughput.

Vaccines against hepatitis B virus have been developed some years ago. For vaccination the envelope protein of the virus (HBsAg) or parts thereof are used. Successful vaccination results in induction of antibodies against HBsAg (anti-HBs). Since the core antigen HBcAg is not part of the vaccines, no anti-HBc antibodies (anti-HBcAg) are generated by vaccination. In contrast, HBV infection nearly invariably induces anti-HBcAg. Therefore, individuals with prior or ongoing HBV infection can be discriminated from healthy vaccinees by the presence of anti-HBcAg. Anti-HBcAg is therefore routinely used for the screening of blood donations; anti-HBcAg positive sera are excluded.

A frequently observed problem in testing blood samples immunologically is that for certain samples the test results are not clearly positive or clearly negative. In such cases it remains ambiguous whether the donor has or has had an infection with hepatitis B virus and must be excluded, or whether the individual has been vaccinated and thus is suitable as a donor. Currently, blood donations giving ambiguous anti-HBcAg values cannot be used for the preparation of blood products. On the other hand blood is very precious and should be discarded only when truly contaminated with hepatitis B virus.

It is therefore desirable to improve the test method by a simple and reliable diagnostic confirmation method to define the true immunological status of blood samples.

### SUMMARY OF THE PRESENT INVENTION

A method for detecting immunologically an infection of hepatitis B virus is disclosed wherein antibodies in a body fluid of a patient against an antigen of hepatitis B virus are immunologically detected, whereby a preincubation with highly purified recombinant hepatitis B core antigen is performed. The method of the present invention is a confirmation test that is preferably applied when ambiguous test results are obtained.

The present invention provides a confirmation test, which allows a safe discrimination of all those samples whereby the result of the immunological standard test for detecting antibodies against hepatitis B virus core antigen, is ambiguous. Testing of blood donations for hepatitis B virus is required by law or at least highly recommended in transfusion medicine in order to detect potentially infectious blood donors. Such testing is also advisable in patients undergoing immunosuppressive therapy to prevent reactivation.

Since current commercial anti-HBc assays often generate divergent results, the specificity of such test results is at least unclear. One approach to resolve such unclear results is to retest the sample with a different method. The disadvantage of such alternative testing is that frequently another laboratory has to be used which causes substantial delay and additional costs.

The present invention provides therefore a simple and reliable confirmation test that can easily be adapted into routine practice at low costs. The confirmation assay of the present invention is based on the idea that the testing of samples which show ambiguous results is repeated twice whereby once the test is performed using the routine methods and secondly the same test is performed with the addition of highly purified rHBcAg which is used for preincubation. The preferably used capsid-like particles contain all immunodominant epitopes of natural HBcAg. Antibodies against the core antigen bind to the rHBcAg during preincubation of the sample. Subsequently such antibodies cannot react with the antigen bound to the solid phase or with the competitive antigen of the immunoassay leading to negative results. In case of an unspecific reactivity there will be no change after preincubation with the antigen. The test result can therefore be interpreted as follows:

If after the addition of the capsid-like particles (HBcAg) no antibodies against HBV can be detected in the serum, the test result will be positive. If, however, the preincubation with rHBcAg does not change the result, it can be concluded that the detected reactivity is due to unspecific binding. The ambiguous test result is consequently not caused by HBV but caused by other unspecific causes contained within the serum. Whether the test result goes up or down in case there is a specific reactivity with the capsid-like particles depends on the assay format.

The assay principle is that rHBcAg specifically traps anti-HBcAg antibodies in serum samples that score positive in commercial anti-HBcAg assays. A sample scoring positive because of non-specific reactivity with components of the anti-HBcAg assay will not be influenced by rHBcAg; for a truly anti-HBcAg positive the added rHBcAg will trap the anti-HbcAg antibodies and influence the read-out.

For specificity of the confirmatory assay, it is therefore mandatory that the antigenicity of the rHBcAg used be as closely as possible identical to that of genuine viral HBcAg. Such antigenic identity is strongly affected by the quality of the rHBcAg preparation. The preparation must not contain non-assembled or partially or totally misfolded core protein subunits. This is warranted best by rHBcAg from full-length HBc rather than truncated variants; the packaged RNA present in particles from full-length core protein particles but not from CTD-truncated variants provides additional stability for the assembled particles [Birnbaum & Nassal (1990), J. Virol., pp. 3319-3330]. This is further exemplified by the relative ease with which particles from CTD-less core protein can be disassembled under mildly denaturing conditions whereas particles from full-length core protein remain stable unless strong denaturants such as SDS are used; these conditions, however, also unfold the individual subunits. Only recently has a disassembly/reassembly procedure been developed for RNA-containing full-length core protein particles, an essential part of which is treatment with 7 M guanidinium hydrochloride [Porterfield et al (2010), J. Virol. pp. 7174-7184].

The improvement obtainable by the method of the present invention strongly depends on the quality of the recombinant hepatitis B core antigen. Therefore, a process for producing suitable HBcAg has been developed.

It will be understood by those skilled in the art that one or more aspects of this invention can meet certain objectives, while one or more other aspects can meet certain other objectives. Each objective may not apply equally, in all its respects, to every aspect of this invention. As such, the inventive objects noted herein can be viewed in the alternative with respect to any one aspect of this invention.

It will also be understood that both the foregoing summary of the present invention and the following detailed description are of exemplified embodiments, and not restrictive of the present invention or other alternate embodiments of the present invention. Other objects and features of the invention will become more fully apparent when the following detailed description is read in conjunction with the accompanying figures and examples. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below, and will be readily apparent to those skilled in the art. Such objects, features, benefits and advantages will be apparent from the above in conjunction with the accompanying examples, data, figures and all reasonable inferences to be drawn therefrom, alone or with consideration of the references incorporated herein.

### BRIEF DESCRIPTION OF THE DRAWINGS

Various aspects and applications of the present invention will become apparent to the skilled artisan upon consideration of the brief description of the figures and the detailed description of the present invention and its preferred embodiments that follows:

FIG. 1: Depicts the results of the confirmatory assay using the Siemens ADVIA Centaur XP System.

FIG. 2: Depicts the results of confirmatory assay using Abbotts Architect i1000.

FIG. 3: Depicts the percentage of inhibition in anti-HBc-positive (n=55) and anti-HBc-negative (n=30) sera measured in order to show the significance of the confirmation test of the present invention. Differences between groups are highly significant (Mann-Whitney test, p<0.001).

### DETAILED DESCRIPTION OF THE PREFERRED EMBODIMENTS

Although any methods and materials similar or equivalent to those described herein can be used in the practice or testing of embodiments of the present invention, the preferred methods, devices, and materials are now described. However, before the present materials and methods are described, it is to be understood that the present invention is not limited to the particular sizes, shapes, dimensions, materials, methodologies, protocols, etc. described herein, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which the present invention belongs. In case of conflict, the present specification, including definitions, will control. In addition, the materials, methods, and examples are illustrative only and not intended to be limiting.

Hepatitis B virus (HBV), the causative agent of acute and chronic hepatitis B, is a small enveloped DNA containing virus. Its icosahedral nucleocapsid, commonly termed core particle, is formed by multiple copies of a single capsid (“core”) protein consisting of 183 or 185 amino acids (depending on HBV subtype). The protein forms stable dimers, 90 or 120 of which assemble to core particles. Structurally similar particles form, in the absence of other viral gene products, upon expression of the viral C gene in heterologous hosts, including bacteria. Using an early bacterial expression system, Nassal had established that the core protein consists of two domains; the first about 140 amino acids are required for assembly into particles (“assembly domain”), the sequence downstream of aa 149 (C terminal domain/CTD) is highly basic and serves as nucleic acid binding domain (Birnbaum & Nassal, 1990).

Based on these data, high resolution cryo EM [Böttcher et al. (2006) J. Mol. Biol., pp. 812-822] and eventually a 3.5 Å x-ray crystallographic 3D structure have been derived from bacterially expressed core protein 1-149. Upon expression of the complete core protein sequence in bacteria, the CTD leads to non-sequence-specific encapsidation of RNA (3000-4000 nt per particle); in appropriate eukaryotic cells in the context of the full genome the CTD is required for specific co-packaging of the viral pgRNA (3.500 nt) and the viral polymerase. The initially formed pgRNA containing nucleocapsids are then converted by the reverse transcriptase activity of the polymerase into DNA containing, mature nucleocapsids which are enveloped and secreted as infectious virions.

Genuine viral core particles are extremely immunogenic and induce a usually life-long antibody response; serologically, core particles are termed hepatitis B core antigen (HBcAg). Recombinant HBcAg (rHBcAg) shares important epitopes with genuine rHBcAg but whether it is 100% identical in antigenicity is not clear; the currently best direct structural data show only subtle, minor differences between RNA-containing E. coli derived capsids and authentic, virion-derived nucleocapsids. It seems that at least early preparations of rHBcAg often responded differently from genuine HBcAg, and variably, against anti-HBcAg. The most likely explanation is structural heterogeneity of these preparations, regarding the assembly status (presence of non-assembled subunits) and possibly the folding status of individual subunits.

The importance of folding and assembly is emphasized by the existence of a natural, non-assembling variant of the core protein that is serologically defined as HBeAg.

The viral C gene encoding the core protein is preceded by the in-frame preC open reading frame (ORF). Translation of the joint preC/C gene yields the so-called precore protein which is a core protein containing 29 additional aa at the N terminus. The first 19 of these amino acids act as a cleavable signal sequence that directs the precore protein into the cell's secretory pathway. By additional processing the CTD region is removed. The end product contains the assembly domain of the core protein, preceded by 10 aa from the preC region. HBeAg is not yet well characterized biophysically but clearly it does not assemble into particles and is antigenically distinct from HBcAg. One established reason for the distinct antigenicities is the assembled state of HBcAg vs. non-assembled of HBeAg. In the closed icosahedral shell of HBcAg some epitopes are physically hidden inside the particle structure but solvent-exposed in non-assembled HBeAg. This is documented by the use of dissociated (by partial denaturation) rHBcAg particles as surrogate positive control for natural HBeAg in some commercial HBeAg ELISAs.

In addition, the fold of the subunits may differ, generating distinct conformational epitopes such that even if the corresponding parts of the protein chain may be solvent-exposed on HBcAg particles and HBeAg, they react differentially with anti-HBcAg and anti-HBeAg antibodies. This second potential reason for distinct antigenicity of HBcAg vs. HBeAg is not well documented because the structure of HBeAg is not known, except that it contains an intramolecular disulfide bridge that is not formed in HBcAg.

A bacterial rHBcAg expression system has been established and purification procedures for rHBcAg, including from full-length core protein, that (as far as possible) meet the criteria for yielding stable, intact rHBcAg particles with (near) authentic HBcAg reactivity.

In one aspect the present invention provides a process for preparing rHBcAg particles, which are required for the confirmation test. The subunits are composed of proteins having the SEQ ID NO:1.

In a preferred embodiment the gene coding for the core particles has SEQ ID NO:2. The gene is inserted into a bacterial expression vector. Several expression vectors can be used. Preferred are vectors as described in the examples.

The expression of the particles is preferably performed in bacteria. In particular preferred is E. coli, whereby E. coli strain BL21 is especially preferred.

After the host has been genetically modified by inserting the gene, the bacteria are induced.

An important aspect of the present invention is the proper purification of the rHBcAg particles. It is particularly preferred to purify the particles by a centrifugation at about 180000-220000 g for 1-3 hours by using a sucrose step gradient ranging preferably from 10% sucrose to 60% sucrose.

Hereinafter, the present invention is described in more detail with reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

### Example 1

### Bacterial rHBcAg Expression Vectors

The aa sequence of the core protein is identical to aa 1-183 of an HBV clone of proven infectivity of genotype D, HBsAg subtype ayw3, GenBank accession number J02203. It is encoded by a synthetic gene (GenBank accession number M20706) carrying multiple nucleotide exchanges that are silent on the protein level. The amino acid sequence corresponds to SEQ ID NO:1, the nucleic acid sequence coding for the gene corresponds to SEQ ID NO:2.

After initial work using bacteriophage lambda pL promoter-based vectors yields of rHBcAg were improved by generation of bacteriophage T7 promoter-based vectors (pET28a2-HBc183; conferring ampicillin resistance [Vogel et al (2005), Proteins, pp. 478-488]; or analogous pET28a-HBc183, conferring kanamycine resistance.

### Example 2

### Expression of rHBcAg

### a) Expression Hosts

Initially, E. coli strain BL21<DE3> (providing T7 RNA polymerase for transcription from the T7 promoter on the pET-derived vectors) was used for rHBcAg expression. This worked well for expression of CTD-truncated HBc but less well for full-length HBc1-183 expression. The reason is the accumulation of Arg-residues in the CTD that are mostly encoded by Arg codons that are rarely used in E. coli. This shortcoming was overcome by using E. coli BL21<DE3> derivatives carrying an extra plasmid providing rare E. coli tRNAs (Codonplus, pRARE) and conferring chloramphenical resistance.

### b) Induction Conditions

General induction conditions are described in [Vogel et al., FEBS Lett. (2005), pp. 5211-5216]. Expression at lower temperature (22-27° C.) generally yields less total HBc protein than expression at 37° C., however, more of the protein is present in the form of intact particles.

### Example 3

### Purification of rHBcAg Particles

The HBcAg expressing bacteria were lysed as follows. In brief, the frozen cell pellets were treated with lysozyme to break the cell wall, benzonase (alternatively RNase+DNase I) to digest bacterial DNA and RNA (RNA packaged in particles is protected; and sonication. This step is crucial as too harsh sonication may cause particle dissociation or even denaturation of the subunits. Good separation of particles from non-assembled proteins is then achieved by sedimentation velocity in sucrose step gradients (10% to 60% sucrose in TN300 buffer (25 mM Tris-HCl, pH 7.5, 300 mM NaCl); the high NaCl concentration stabilizes the particles as evidenced by the fact that deliberate dissociation HBc particles requires, amongst other conditions such as high pH and denaturing agents such as urea, very low salt to be effective; conversely, re-assembly can be initiated by reducing the denaturing agent and increasing the salt concentration.

The size of the gradient tubes and rotor depends on the scale of preparation. Typical for a bacterial culture of 200-250 ml is use of a Kontron TST41.14 rotor (or equivalent from another supplier) run at 41,000 rpm at 20° C. for 2 h, average RCF 200,000 g. Run conditions for other rotors can be calculated from the rotor geometry. Under these conditions, intact particles (s-value=˜80S) typically sediment into the center of the gradient, running ahead of ribosomal subunits; non-assembled HBc (usually very little for HBc1-183 particles) and E. coli proteins remain in the upper gradient fraction. Misfolded or otherwise aggregated proteins run ahead of the particles.

If necessary (e.g. if too much lysate was loaded on a gradient, resulting in contamination of the rHBcAg with E. coli proteins), the proper gradient fractions can be pooled, dialyzed against TN300 buffer, and subjected to a second gradient run under identical conditions. Smaller amounts of rHBcAg from a single gradient, or larger amounts from two sequential gradients are routinely ≧95% pure as judged by SDS-PAGE analysis and appearance of a single 21 kDa band upon Coomassie-Blue staining.

For some applications, it may be desired to remove non-proteinaceous contaminants (not visible by Coomassie-Blue staining after SDS-PAGE), e.g. E. coli membrane components including LPS. This can be achieved by phase separation using Triton X114 detergent. Membrane components accumulate in the detergent-rich phase, rHBcAg remains in the low-detergent phase. A specific procedure would be to use X-114 partitioning on rHBcAg isolated from a first sucrose gradient as described above, followed by resedimentation on a second gradient which further purifies the rHBcAg particles and removes remaining detergent.

### Example 4

### Proof for Intact Particle Nature of rHBcAg

### 4.1 Sedimentation Velocity

A first important indication for intact particles is sedimentation behaviour in the above described gradients. Material not sedimenting into the gradient center does not represent intact particles, material in the gradient center is highly likely to represent particles. Additional proof can be obtained by alternative methods.

### 4.2 Native Agarose Gel Electrophoresis (NAGE)

Due to their large size, intact particles can not enter polyacrylamide gels but do so when 1%-2.5% agarose is used as electrophoresis matrix. Mobility depends on the physical state of the protein and surface charge. Due to the large pore size of agarose, diffusion of non-assembled subunits is much faster than that of assembled particles; particles therefore produce much more distinct bands. Aggregates, on the other hands, may be too large to enter even the agarose gel and remain in the loading slot. Misfolded smaller aggregates may enter the gel, but are heterogeneous in size and surface charge, usually resulting in a broad smear. Hence the decisive criterion for intact particles is formation of a distinct band.

A second criterion for particles from full-length HBc is the appearance of distinct fluorescent band when the NAGE is run in the presence of a nucleic acid stain such as ethidium bromide. The RNA in E. coli derived rHBcAg is heterogeneous in size and would give rise to a broad smear if not encapsidated. However, if encapsidated, all RNA molecules reside in the particle interior and are transported in the electric field together with the capsid protein. Hence the corresponding band stains with the RNA stain and also with protein stain. Furthermore, the work-up procedure includes treatment with nuclease(s) which degrade(s) non-protected RNA and DNA. The presence of RNA at the same position in the NAGE gel as that of capsid protein also demonstrates that these RNA molecules must be protected by an intact capsid shell, otherwise they would have been degraded by the added nuclease(s).

### 4.3 Electron Microscopy

Several studies have shown that rHBcAg isolated according to the procedures outlined above is present as regular, genuine HBcAg like particles in negative staining EM and, at higher resolution, in 3D reconstructions based on cryo EM.

### 4.4 Absence of HBeAg-Antigenicity

An additional quality control—not routinely performed—is to test the rHBcAg preparation for reactivity with anti-HBeAg antibodies. Such reactivity should be very low.

### Example 5

### Use of Intact Particles rHBcAg in Confirmatory Assays

The method of the present invention has been performed by using widely used HBV test systems whereby the rHBcAg particles as described herein were used in the confirmation test.

### 5.1 Confirmatory Assay Using the Siemens ADVIA Centaur XP System

185 clinical samples were analysed by the confirmatory assay using the Siemens system. 31 of these samples had been sent by external laboratories for confirmation of low positive results in other assay systems. They were all confirmed reactive. The remaining samples were tested because of low reactivities in the Centaur HBc Total assay. 17 of these samples were not inhibited by the recombinant antigen, in 3 cases the percentage of inhibition was in the defined grey zone of the assay (Cut-off minus 20%). Anamnesis data and follow-up samples were not helpful for the resolution of these cases.

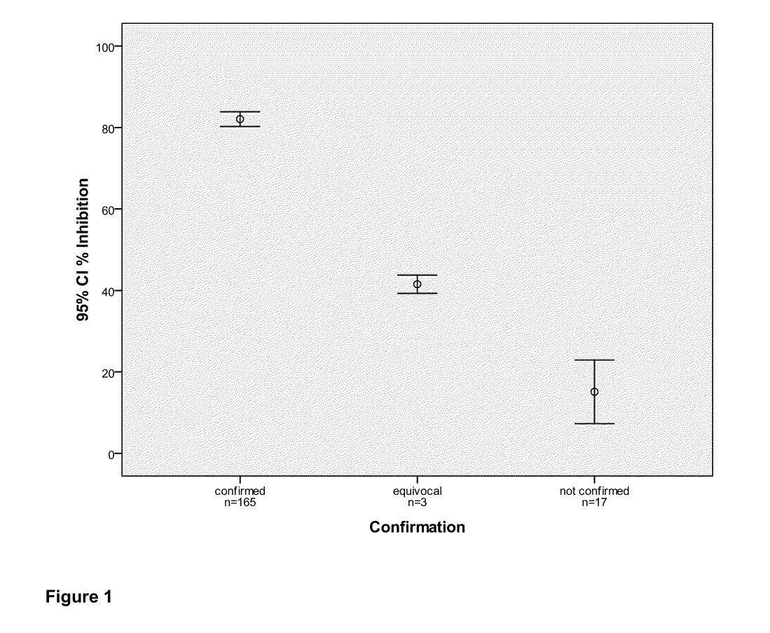

Results of the confirmatory assay using the Siemens ADVIA Centaur XP System are shown in FIG. 1.

As FIG. 1 shows, 165 out of 185 samples had been confirmed. This corresponds to nearly 90%. 17 out of 150 samples have not been confirmed. This corresponds to about 9%. Only 3 samples out of 185 (corresponding to 1.62%) remained unclear.

5.2 Confirmatory Assay Using Abbotts Architect i1000

44 clinical samples were analysed by Abbott Architect i1000. 24 out of these were confirmed, 2 were equivocal and 18 could not be confirmed. 14 out of these samples had been tested positive in other laboratories using the Abbott Architect i2000. 4 of the 14 external samples were confirmed reactive, while 10 were not confirmed. All unconfirmed samples were also negative in the Siemens Centaur system.

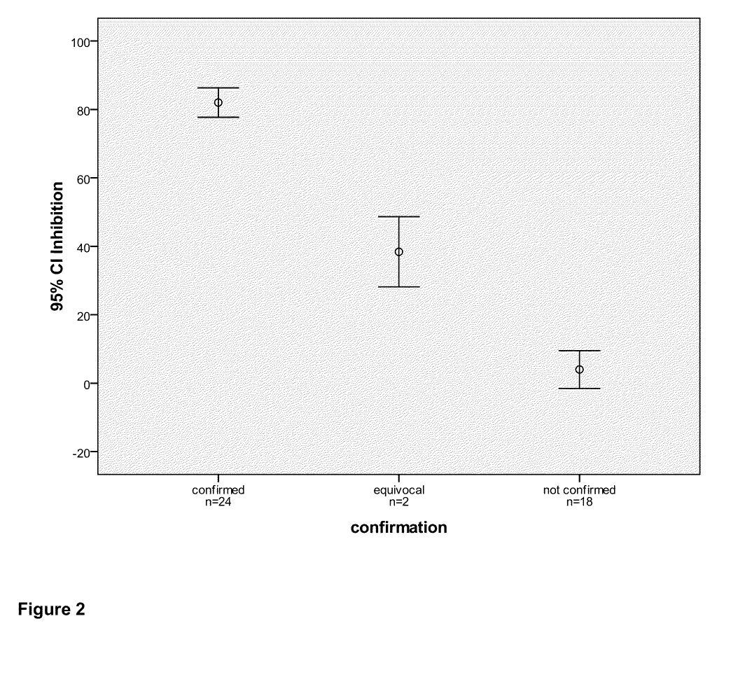

Results of the confirmatory assay using Abbotts Architect i1000 are shown in FIG. 2.

75 samples had been sent by the department of transfusion medicine because of reactivity in the Abbott Axsym system. They were all analysed by Centaur HBcT and Architect Anti-Hbcll. 34 out of these samples were reactive in one or both assay systems; 29 out of these were confirmed positive, in five cases, the confirmatory assay was negative. 42 samples were clearly negative in both assay systems, so the confirmatory assay could not be applied.

12 out of the 29 true positive samples were from repeat donors and a look-back of earlier samples was done. Only in one donor seroconversion was confirmed.

Seven of the confirmed positive donors had been tested negative by Abbott Architect, four were negative by Siemens Centaur.

Two out of the 29 confirmed cases were anti-HBc only cases (anti-HBs negative). In 5 out of 29 Anti-HBs was below 100 IU/I, in 22 above 100 IU/I.

### 5.3 Evaluating the Inhibition

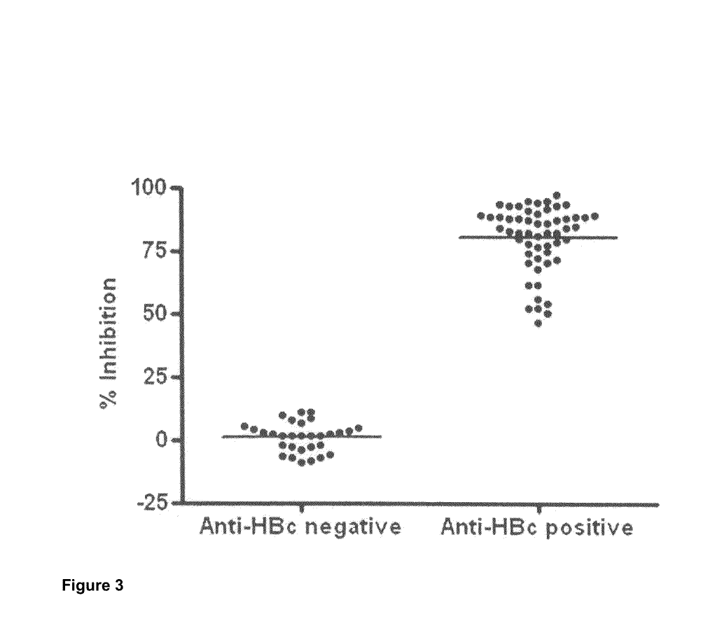

In order to show the significance of the confirmation test of the present invention the percentage of inhibition in anti-HBc-positive (n=55) and anti-HBc-negative (n=30) sera has been measured. The result is shown in FIG. 3. The differences between the groups are highly significant (Mann-Whitney test, p<0.001). FIG. 3 shows that the test results are reliable and that the confirmation test as disclosed herein allows a clear diagnosis of ambiguous samples at low cost.

5.4 Selection of Suitable Concentration of rHBcAg

A concentration of 1 μg/ml rHBcAg was sufficient to inhibit more than 50% reactivity in all three commercial tests. Preincubation temperature had no influence on the inhibition and there was no difference in signal intensity, if PBS was used instead of negative serum as dilution matrix.

Subsequent analyses were thus done using rHBcAg in PBS, the final concentration of 1 μg/ml for inhibition and PBS only for the control reaction. In order to demonstrate the capacity of the rHBcAg as used in the present invention to inhibit antibodies induced by different HBV genotypes sera from patients with chronic HBV infection and known HBV genotype were analyzed. The results are shown in Table 1.

TABLE 1

| | | | | |
|---|---|---|---|---|
| | | ADVIA | | |
| | | Centaur ® | Architect ® | |
| | | HBcT | anti-HBcII | |
| | | PBS/antigen | PBS/antigen | LIAISON ® |
| Sample | Dilution | (% inhibition) | (% inhibition) | anti-HBc |

PBS/antigen (% inhibition)

| | | | | |
|---|---|---|---|---|
| Genotype A | 1:50000 | 76212/10448 | 44705/3796 | 6729/63564 |
| | | (86.3%) | (91.5%) | (89.4%) |
| Genotype | 1:500 | 23690/10831 | 84894/54313 | 3573/34186 |
| C-1 | | (54.3%) | (36%) | (89.5%) |
| | 1:5000 | 7972/3511 | 16719/2091 | 6864/63673 |
| | | (56%) | (87.5%) | (89.2%) |
| Genotype | 1:5000 | 37528/20048 | 49868/18984 | 8437/53485 |
| C-2 | | (46.6%) | (61.9%) | (84.2%) |
| | 1:50000 | 11085/3111 | 7601/1285 | 36964/74180 |
| | | (71.9%) | (83.1%) | (50.2%) |
| Genotype | 1:5 | 109168/7757 | 45848/9736 | 7100/70849 |
| D-1 | | (92.9%) | (78.8%) | (90%) |
| Genotype | 1:50 | 122923/22929 | 64866/7686 | 2185/32918 |
| D-2 | | (81.3%) | (88.2%) | (93.4%) |
| Genotype E | 1:500 | 229293/64167 | 103208/49436 | 1040/14560 |
| | | (72%) | (52.1%) | (92.9%) |
| | 1:5000 | 32680/6604 | 23436/9031 | 15466/66771 |
| | | (79.8%) | (61.5%) | (76.8%) |

The disclosure of each publication, patent or patent application mentioned in this specification is specifically incorporated by reference herein in its entirety. However, nothing herein is to be construed as an admission that the invention is not entitled to antedate such disclosure by virtue of prior invention.

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method for confirming an infection of hepatitis B virus in a patient, said method comprising the steps of:
a. immunologically detecting antibodies against an antigen of hepatitis B virus in a body fluid of said patient,
b. repeating step (a) at least once under substantially identical conditions using the same immunological test, wherein one of steps (a) or (b) is performed with a preincubation with HBcAg and the other is performed without preincubation with HBcAg, and
c. comparing the results obtained in steps (a) and (b), wherein differing results in steps (a) and (b) indicates that the patient is infected with hepatitis B virus.

2. The method according to claim 1 wherein the body fluid is serum.

3. The method according to claim 1, characterized in that the HBcAg as used for the preincubation with the serum is a recombinantly produced HBcAg preparation (rHBcAg) that contains all immunodominant epitopes of genuine viral HBcAg.

4. The method according to claim 3, characterized in that the preparation contains less than 10% non-assembled or partially or totally misfolded core protein subunits.

5. The method according to claim 3 wherein the HBcAg is recombinantly produced in E. coli.

6. The method according to claim 5, characterized in that the recombinantly produced HBcAg consists of subunits having the amino acid sequence shown in SEQ ID NO:1.

7. The method according to claim 5, characterized in that the rHBcAg is used in the form of intact particles.

8. The method according to claim 7, characterized in that the intact rHBcAg particles are recombinantly produced in E. coli strain BL 21.

9. The method according to claim 5, characterized in that the gene coding for the amino acid sequence according to SEQ ID NO:1 is encoded by a DNA sequence having SEQ ID NO:2.

10. The method according to claim 5, characterized in that the recombinantly produced HBcAg particles are purified by centrifugation in sucrose step gradient.


SEQUENCE LISTING (2 sequences)

SEQ ID NO: 1 - protein, 183 aa, Hepatitis B virus
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 2 - DNA, 556 bp, Hepatitis B virus
catggatatcgatccttataaagaattcggagctactgtggagttactctcgtttctcccgagtgacttctttccttcagtacgagatcttctggataccgccagcgcgctgtatcgggaagccttggagtctcctgagcactgcagccctcaccatactgccctcaggcaagcaattctttgctggggggagctcatgactctggccacgtgggtgggtgttaacttggaagatccagctagcagggacctggtagtcagttatgtcaacactaatatgggtttaaagttcaggcaactcttgtggtttcacattagctgcctcactttcggccgagaaacagttctagaatatttggtgtctttcggagtgtggatccgcactcctccagcttataggcctccgaatgcccctatcctgtcgacactcccggagactactgttgttagacgtcgaggcaggtcacctagaagaagaactccttcgcctcgcaggcgaaggtctcaatcgccgcggcgccgaagatctcaatctcgggaatctcaatgttagtga